(19) United States
(12) Patent Application Publication
(10) Pub. No.: US 2015/0017185 A1
(43) Pub. Date: Jan. 15, 2015

(54) METHOD TO IMPROVE THE IMMUNE FUNCTION OF T CELLS

(71) Applicant: UCL BUSINESS PLC, London (GB)

(72) Inventors: Arne Akbar, London (GB); Sian Henson, London (GB)

(21) Appl. No.: 14/361,604

(22) PCT Filed: Nov. 29, 2012
(86) PCT No.: PCT/GB2012/052948

§ 371 Date: May 29, 2014

(30) Foreign Application Priority Data
Nov. 29, 2011 (GB) 1120527.5

(51) Int. Cl. C07K 16/28; A61K 31/5377; A61K 39/395; C12N 5/0783

(52) U.S. Cl. CPC C07K 16/2827; C12N 5/0638; A61K 31/5377; A61K 39/3955; C12N 2501/51; C12N 2501/727
USPC 424/173.1; 435/375

(57) ABSTRACT

The present invention provides a method for enhancing the immune function of a memory T cell which comprises the step of coinhibting signalling via an inhibitory receptor which regulates T cell exhaustion and via the p38 MAP kinase signalling pathway in the T cell, and a method for treating and/or preventing an immune condition in a subject, which comprises the step of enhancing the immune function of a memory T cell in the subject by such a method. There is also provided a pharmaceutical composition or kit comprising an agent capable of inhibiting signalling via an inhibitory receptor which regulates T cell exhaustion, such as PD-1, and an agent capable of inhibiting the p38 MAP kinase signalling pathway.

### FIELD OF THE INVENTION

The present invention relates to methods and compositions for enhancing the immune function of T cells, and the use of such methods and compositions to treat certain immune conditions, often associated with aging.

### BACKGROUND TO THE INVENTION

The immune system undergoes dramatic re-structuring with age. There is a marked decline in the number of naïve T cells produced by the thymus that results from thymic atrophy. The reduced thymic T cell production necessitates that memory T cell pool is maintained by continuous proliferation. The lifelong re-challenge, especially with persistent antigen, leads to the accumulation of highly differentiated T cells that are frequently expanded and this is associated with a decline in immune responsiveness. These changes are collectively referred to as immune senescence which is associated with an increase in the frequency and severity of infections, a higher incidence of malignancy and decreased responses to vaccination in older adults.

Highly differentiated CD8⁺ T cells also accumulate in patients with malignancy and those with persistent infections, such as HIV, EBV, CMV, HBV and HCV.

It would be highly desirable to improve or restore functionality in these T cells in order to treat immune conditions, particularly in older subjects, and to enhance responses during immunotherapeutic regimes such as vaccination.

Human T cells at late stages of differentiation can be identified by the loss of surface markers CD27, CD28 and CCR7 and also the re-expression of CD45RA. Furthermore, highly differentiated memory T cells have relatively short telomeres and have changes in cell signalling pathways including defective Akt/PKB and increased p38 MAP kinase (MAPK) phosphorylation that are associated with cellular senescence. These changes are particularly evident in highly differentiated effector memory T cells that re-express CD45RA (EMRA T cells) which are considered to be an end-stage effector population. The EMRA CD8⁺ T cell population also exhibits functional changes including the loss of proliferative and telomerase activity and have increased susceptibility to apoptosis.

Two different cellular processes can lead to T memory cell dysfunction. First, repeated antigenic stimulation of T cells may induce a state of functional exhaustion where proliferative activity and cytokine production are lost. The inhibitory receptor programmed death 1 (PD-1), a negative regulator of activated T cells, is upregulated on the surface of exhausted CD8 T cells during some chronic viral infections such as HIV and HCV (Day et al (2006) Nature 443:350-4; Trautmann et al (2006) Nat Med 12:1198-202; and Urbani et al (2006) J. Virol 80:11398-403). Blockade of the PD-1/PD-1 ligand (PD-L1) has been shown to restore CD8 T cell function and reduce viral load (Day et al (2006) as above).

The second cellular process can lead to T memory cell dysfunction is repeated T cell stimulation which can induce replicative senescence; where cells lose proliferative potential through loss of telomerase activity, telomere erosion and increased susceptibility to apoptosis; but remain functional. It has recently been shown that the blockade of p38 signalling in highly differentiated primary human CD4⁺ T cells can increase their telomerase activity (Di Mitri et al., 2011 J. Immunol 187).

### DESCRIPTION OF THE FIGURES

FIG. 1. CD8⁺ EMRA cells exhibit characteristics of senescent T cells.

Representative example of CD45RA and CD27 staining on CD8⁺ T cells (A). Data showing the expression of CD57 on CD8⁺ CD45RA/CD27 T cell subsets ex vivo, horizontal lines depict mean values (B). Data showing the loss of telomere length within CD45RA/CD27 CD8⁺ T cells, horizontal lines depict mean values (C). Representative example and pooled data of γH2AX staining on CD45RA/CD27 subsets following a 4 day stimulation with 0.5 μg/ml anti-CD3 and 5 ng/ml IL-2, graph shows the mean±SE for 4 donors (D). The analysis has been performed on non-proliferating lymphocytes. Data showing phosphorylated p38 levels following a 20 minute incubation with 50 ng/ml PMA and 500 ng/ml ionomycin in CD8⁺ CD45RA/CD27 T cell subsets (E). Data has been normalised in relation to the CD45RA⁺CD27⁻ subset, the graph shows the mean±SE for 9 donors and P values have been calculated using t-tests. Representative immunoblots of phosphorylated and total p38 together with β-actin for CD8⁻ CD45RA/CD27 subsets examined directly ex vivo (F). All P values were calculated using the Student's t-test.

FIG. 2. Expression of PD-1 during CD8⁺ T cell differentiation.

Representative example and data of PD-1 expression on CD8⁻ CD45RA/CD27 defined T cell subsets. Horizontal lines depict mean values and P values were calculated using the Student's t-test.

FIG. 3. Differentiation-related functional changes in human CD8⁺ T cells.

Data showing the proliferation of CD45RA/CD27 subsets assessed by Ki67 staining following stimulation 0.5 μg/ml anti-CD3 and irradiated APCs for 4 days, horizontal lines depict mean values (A). Representative example and graph showing telomerase activity in CD8⁺ CD45RA/CD27 subsets following a 4 day incubation with 0.5 μg/ml anti-CD3 and irradiated APCs, graph shows the mean±SE for 4 donors (B). Multiparameter flow cytometry examining the expression of IFNγ. TNFα, perforin and granzyme B in CD8⁺ CD45RA/CD27 T cell subsets following an 8 hour stimulation with 0.5 μg/ml anti-CD3. Pie charts show the average of 7 donors (C). Data showing the percentage of CD8⁺ CD45RA/CD27 T cell subsets containing perforin and granzyme(C). The data was generated following an 8 hour stimulation with 0.5 μg/ml anti-CD3. Graph showing the expression of CD107a in CD8⁺ CD45RA/CD27 T cell subsets following an 8 hour stimulation with 0.5 μg/ml anti-CD3, horizontal lines depict mean values (E). All P values were calculated using the Student's t-test.

FIG. 4. Signalling through both PD-1 and p38 pathways contribute to reduced proliferation the of CD8⁺ EMRA T cells.

Representative example of Ki67 staining on CD45RA⁺CD27⁻ T cells measured after 4 days stimulation with 0.5 μg/ml anti-CD3 and irradiated autologous mononuclear cells (A). This activation was performed in the presence of 10 μg/ml anti-PDL1/2 antibodies or 500 nM BIRB796. In control cultures, 10 μg/ml IgG2a, IgG2b or 0.1% DMSO were added individually or together. Pooled data showing the effect of PDL1/2 antibody block. BIRB7 96 or both molecules on proliferative, measured by Ki67, in CD8⁺ CD45RA/CD27 T cell subsets that were activated as above (B). The graph shows the mean±SE for 4 donors and P values were calculated using paired t-tests.

FIG. 5. Signalling through p38 but not PD-1 pathways regulates reduced telomerase activity of CD8⁺ EMRA T cells.

Representative blot for telomerase activity in CD8⁺ CD45RA/CD27 T cell subsets on day 4 following activation with 0.5 μg/ml anti-CD3 and irradiated autologous APCs (A). The blot is representative of 4 separate experiments. Pooled data examining telomerase activity following incubation with 10 μg/ml anti-PDL1/2, 500 nM BIRB 796 or both molecules after 4 days of stimulation (B). The graph shows the mean±SE for 4 donors and P values were calculated using paired t-tests.

FIG. 6. Signalling through p38 inhibits TNF-α secretion that is reversed by inhibiting the PD-1 pathway in CD8⁺ T cell subsets.

Graph showing the production of TNFα, by CD8⁺ CD45RA/CD27 subsets following a 48 hour stimulation with 0.5 μg/ml anti-CD3 and 5 g/ml IL-2 with either 10 μg/ml anti-PDL1/2 block, 500 nM BIRB796 or both molecules. The graph shows the mean±SE for 5 donors and P values were calculated using paired t-tests.

FIG. 7. CD45RA⁺CD27⁺ CD8⁺ T cells account for a large proportion of resident skin T cells.

Immunofluorescence staining showing CD4 (left panel) and CD8 (right panel) in representative normal skin biopsies (A). The graph shows the ratio of CD4:CD8 T cells, horizontal line depicts mean values. Double immunofluorescence staining of representative biopsies. Green indicates CD45RA; red indicates CD8 (B). The graph shows the percentage of CD8⁺ cells expressing CD45RA, horizontal lines depict mean values. Original magnification for all images, ×400 and cell numbers were expressed as the mean absolute number of cells counted within the frame.

FIG. 8. Further characteristics of CD8⁺ EMRA T cells.

A. Representative example of γH2AX and Ki67 staining on CD8+ T cells gated on non-proliferating and proliferating cells defined by the forward and side scatter flow cytometry profile. Data obtained following a 4 day stimulation with 0.5 μg/ml anti-CD3 and 5 ng/ml IL-2. B. Graph showing cumulative data of multiparameter flow cytometry examining the expression of IFNγ, TNFα, perforin and granzyme B in CD8⁺ CD45RA/CD27 T cell subsets following an 8 hour stimulation with 0.5 μg/ml anti-CD3. The graph shows the mean±SE for 7 donors and P values were calculated using paired t-tests.

### SUMMARY OF ASPECTS OF THE INVENTION

The present inventors have found that, by inhibiting both the senescence and exhaustion pathways simultaneously, highly differentiated T cells can be rejuvenated to exhibit increased proliferative potential and also retain their effector function. This cannot be achieved by blocking either pathway alone. Thus the co-inhibition of both pathways may be used for selective immune enhancement, for example in subjects with defective immunity.

In a first aspect the invention provides a method for enhancing the immune function of a T cell which comprises the step of inhibiting both:
(i) signalling via an inhibitory receptor that regulates T cell exhaustion; and
(ii) the p38 MAP kinase signalling pathway in the T cell.

The inhibitory receptor which regulates T cell exhaustion may, for example, be selected from: PD-1, Tim-3, Lag-3 or CTLA-4.

The method may comprise the step of co-inhibiting signalling via PD-1 and via the p38 MAP kinase signalling pathway in the T cell.

The proliferative potential of the T cell may be enhanced by the method. This may be without significant loss of T cell function. For example, the capacity of the T cell to secrete one or more cytokines, such as TNFα, may be restored or substantially retained by the method.

The telomerase activity of the T cell may be enhanced.

Where the inhibitory receptor that regulates T cell exhaustion is PD-1, PD-1 signalling may be inhibited by blocking the PD-1 ligand (PD-L). PD-L may blocked, for example, by using antibodies against PD-L1 and PD-L2.

The T cell may be a memory T cell, such as an effector memory T cell which expresses CD45RA (EMRA T cell). The T cell may be a CD8+ EMRA T cell.

In a second aspect, the present invention provides a method for treating and/or preventing an immune condition in a subject, which comprises the step of enhancing the immune function of a T cell in the subject by a method according to the first aspect of the invention.

There is also provided a method for enhancing the immune response to vaccination in a subject which comprises the step of enhancing the immune function of a T cell in the subject by a method according to the first aspect of the invention.

In a third aspect, the present invention provides a pharmaceutical composition comprising an agent capable of inhibiting signalling via PD-1, Tim-3, Lag-3 or CTLA-4, and an agent capable of inhibiting the p38 MAP kinase signalling pathway.

In a fourth aspect, the present invention provides a kit comprising:
(i) an agent capable of inhibiting signalling via PD-1, Tim-3, Lag-3 or CTLA-4; and
(ii) an agent capable of inhibiting the p38 MAP kinase signalling pathway for separate, sequential or simultaneous administration.

The pharmaceutical composition according or may be for use in enhancing the immune function of a T cell.

In a fifth aspect, the present invention provides a method for treating and/or preventing an immune condition in a subject, which comprises the step of administering a pharmaceutical composition or a kit according to the invention to the subject.

The immune condition may be associated with aging. For example, the immune condition may be selected from shingles, pneumonia, skin cancer and an immunodeficiency.

### DETAILED DESCRIPTION

### T Cells

The first aspect of the invention relates to a method for enhancing the immune function of a T cell.

The T cell may be in a state of functional exhaustion and/or senescence.

The T cell may be a human T cell at a late stage of differentiation, such as a terminally differentiated T cell. Human T cells at late stages of differentiation can be identified by the loss of surface markers CD27, CD28 and CCR7 and also the re-expression of CD45RA. Although the methods of the present invention are most effective on T cells at a late stage of differentiation, they also work to a lesser extent on T cells at earlier stage of differentiation

The T cell may be a memory T cell, which has previously encountered its cognate antigen, and thus mounts a stronger and faster immune response upon re-exposure to antigen.

The T cell may be a memory T cell which re-express CD45RA (EMRA T cell).

The T cell may be a CD4+ or CD8+ EMRA T cell.

Highly differentiated memory T cells may have changes in cell signalling pathways including defective Akt/PKB and increased p38 MAP kinase (MAPK) phosphorylation. If senescence has been induced in the T cell by a telomere-dependent route (see below) then the T cell may have relatively short telomeres. If, however, the T cell is senescent for telomere-independent reasons, they may have longer telomeres than central memory (CD27+CD45RA−) or EM (CD27−CD45RA−) cells from the same subject.

EMRA CD8⁺ T cell population also exhibits functional changes including the loss of proliferative and telomerase activity and have increased susceptibility to apoptosis.

The T cell may be found in the skin of a subject.

### Immune Senescence and Exhaustion

As explained above, there are two different types of T memory cell dysfunction which are caused by distinct cellular processes.

Functional exhaustion is characterised by a loss of proliferative activity and cytokine production. It may be caused by repeated antigenic stimulation of T cells. Immune exhaustion is initiated by external cell surface inhibitory receptors such as PD-1 and Tim3.

Senescence is characterised by loss of proliferative potential through loss of telomerase activity, telomere erosion and increased susceptibility to apoptosis. Telomeres are repeating hexameric sequences of DNA which are progressively lost with each replicative cycle. In the absence of complementary factors, telomeres shorten by about 50-100 bases after each round of proliferation until the exposed DNA end of the telomere is recognised by the cell as a double-stranded DNA break. This recruits a complex of proteins involved in DNA, known as the DNA damage response (DDR), causing senescence. Cellular senescence can also occur when telomeric or non-telomeric DNA is damaged by means that are independent of telomere shortening. This includes damage by reactive oxygen species, ionizing radiation, chromatin purturbation and activation of p53 and stress pathways.

Senescent T cells express high levels of surface KLRG1 and CD57, and increased levels of the phosphorylated histone γH2AX.

Some senescent T cells, such as CD4+ EMRA T cells are still functional in terms of cytokine secretion, even though they have proliferative defects. They are therefore distinct from functionally exhausted T cells that have proliferative defects but also progressively lose the ability to secrete cytokines and to mediate cytotoxicity.

### Enhancing Immune Function

The present inventors have found that co-inhibition of signalling via PD-1 and via the p38 MAP kinase signalling pathway in a senescent T cell reconstitutes the proliferative potential of the T cell, but also maintains the functional integrity of the T cell, for example, its ability to secrete cytokines.

A T cell with enhanced immune function may thus show enhanced proliferation, for example following TCR and/or IL-2 activation. TCR activation may occur, for example, following presentation of the cognate antigen or epitope, or by using an anti-CD3 antibody.

A T cell with enhanced immune function may show reduced levels of apoptosis, which may be due to upregulation of the anti-apoptotic molecule Bcl-2 in the cells.

A T cell with enhanced immune function may show enhanced telomerase activity and/or an increased capacity to upregulate telomerase activity following T-cell activation.

Secretion of one or more cytokines by the T cell is retained and may even be enhanced. The type of cytokine produced by the T cell will depend on the T cell type as shown in Table 1:

TABLE 1

| T cell type | Cytokines produced |
|---|---|
| Th1 | IL2, IFNγ, IL12, TNFβ |
| Th2 | IL4, IL5, IL6, IL10, IL13 |
| Th3 | TGFβ and IL10 |
| Treg | TGFβ and IL10 |
| Tc | IFNγ, IL2, TNFα |
| TH17 | IL-17, IL-21 and IL-22 |
| TH9 | IL-9 and possibly IL-10 (in |
| | mice only) |
| TH-22 | IL-22 but not IL-17 |

The T cell may be rejuvenated such that it exhibits increased proliferative potential.

The immune function of the T cell may be restored or increased. For example a rejuvenated CD8+ T cell may show restored or increased cytotoxic activity.

### Inhibitory Receptors of T Cell Exhaustion

Various cell surface inhibitory molecules have been found that are involved in the regulation of T cell exhaustion. Gene expression profiles have revealed that exhausted CD8+ T cells may co-express up to seven inhibitory receptors.

The method of the invention comprises the step of inhibiting both:
(i) signalling via a cell surface inhibitory receptor involved in the regulation of T cell exhaustion; and
(ii) the p38 MAP kinase signalling pathway within the T cell.

The cell surface inhibitory receptor involved in the regulation of T cell exhaustion may, for example, be selected from: PD-1, Tim-3, Lag-3 or CTLA-4 (Blackburn et al (2009) Nat. Immunol. 10:29-37).

### PD-1 Signalling

Programmed Death 1, or PD-1, is a Type I membrane protein expressed on the surface of activated T cells, where it acts as an inhibitory receptor. PD-1 is a member of the extended CD28/CTLA-4 family of T cell regulators. The protein's structure includes an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif. SHP-1 and SHP-2 phosphatases bind to the cytoplasmic tail of PD-1 upon ligand binding.

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling. PD-L1 is expressed on almost all murine tumor cell lines. PD-L2 expression is more restricted and is expressed mainly by dendritic cells and a few tumor lines.

PD-1 signalling may be inhibited by blocking PD-L1, PD-L2, or both, for example by using blocking antibodies (Day et al (2006) and Trautman et al (2006) as above).

Alternatively PD-1 activity may be inhibited using small molecules, or PD-1 expression may be inhibited using siRNA.

Similar approached may be employed to block signalling via the other inhibitory receptors involved in T cell exhaustion, such as Tim-3, Lag-3 and CTLA-4.

### P38 Map Kinase Signalling

Mitogen-activated protein kinases (MAPK) are intracellular signalling molecules involved in cytokine synthesis. There are four isoforms of p38: P38α, β, γ and δ, which all share similar activation profiles, but can vary in terms of kinetics and levels of activation. P38α and β are the predominant isoforms expressed in lymphocytes. The p38 MAPK pathway is critical for the production and activity of multiple pro-inflammatory cytokines, including TNFα, IL-1, IL-6 and IL8.

Several small-molecule inhibitors of p38 have been developed, some of which are in clinical trial. Inhibitors include: GSK-681323, SB-85635, AMG-548, AVE-9940, PS-540446, PS-516895, SCIO-469, SC80036, PH-797804, BIRB-796, VX-745, VX-702, RO4402257, RWJ 67675 and TAK-715 (Schindler et al (2007) J. Dent. Res. 86:800).

P38 MAPK signalling may also be inhibited by blocking p38 expression, for example by using siRNA.

### Treating Immune Conditions

The method of the present invention may be used generally to enhance immune function, in order to increase immune surveillance, reduce the frequency and/or severity of infections, and to boost immunity against malignant cells.

The method of the invention may therefore be used to prevent infections such as bacterial, viral, fungal or parasitic infections and/or to treat the immune conditions resulting from such infection.

The method of the invention may also be used to treat an existing cancer.

The second aspect of the invention relates to a method for treating and/or preventing an immune condition in a subject.

The immune condition may be associated with aging.

The immune condition may be shingles, pneumonia, skin cancer or an immunodeficiency.

Shingles is a viral disease characterized by a painful skin rash with blisters which often occurs in a limited area on one side of the body and may be in a stripe. The initial infection with varicella zoster virus (VZV) causes the acute illness chickenpox which generally occurs in children and young people. Once an episode of chickenpox has resolved, the virus is not eliminated from the body and can go on to cause shingles, often many years after the initial infection.

Varicella zoster virus can become latent in the nerve cell bodies and less frequently in non-neuronal satellite cells of dorsal root, cranial nerve or autonomic ganglion, without causing any symptoms. Years or decades after a chickenpox infection, the virus may break out of nerve cell bodies and travel down nerve axons to cause viral infection of the skin in the region of the nerve. The virus may spread from one or more ganglia along nerves of an affected segment and infect the corresponding dermatome causing a painful rash. Although the rash usually heals within two to four weeks, some sufferers experience residual nerve pain for months or years, a condition called postherpetic neuralgia.

Shingles is more common in old-aged people, which is likely to be at least partly due to immune senescence in this population failing to keep the latent virus at bay. Enhancing immune function of senescent memory T cells in these subjects by the method of the invention rejuvenates immune responses and may help treat and/or prevent infection.

Pneumonia is an inflammatory condition of the lung which especially affects the alveoli and is associated with a lack of air space (consolidation) which is visible on a chest X-ray. Pneumonia is typically caused by an infection and infectious agents include bacteria, viruses, fungi, and parasites.

Typical symptoms include cough, chest pain, fever, and difficulty breathing.

Pneumonia is relatively common in elderly and immunocompromised patients (such as HIV positive individuals). Immune senescence or deficiency can mean that the body is unsuccessful in managing or clearing the underlying infection, so that it progresses to pneumonia.

Enhancing immune function of senescent memory T cells in elderly subjects by the method of the invention stimulates immune responses and helps treat the underlying infection, preventing the development of pneumonia.

The pharmaceutical composition may be administered to a subject having an existing infection, such as a bacterial or viral infection, in order to prevent the development of pneumonia.

The method of the invention may also be used to treat a subject who already has pneumonia, in order to resolve the pneumonia. The treatment may also reduce or clear the original infection.

Skin cancer, or skin neoplasms, are skin growths with differing causes and varying degrees of malignancy. The three most common malignant skin cancers are basal cell cancer, squamous cell cancer, and melanoma, each of which is named after the type of skin cell from which it arises. Skin cancer generally develops in the epidermis (the outermost layer of skin) and is caused by exposure to sunlight, particularly the ultraviolet (UV) rays.

Skin cancer is more common in older people, partly because they have had a longer time of exposure to UV rays and partly due to age-related declines in immune function which contribute to an increasing incidence of malignancies (Burns and Leventhal (2000) Cancer Control 7:513-522).

The method of the present invention may be used to enhance immune function in these subjects to prevent and/or treat skin cancers.

Immunodeficiency is a state in which the immune system's ability to fight infectious disease is compromised or entirely absent. Immunodeficiency may also decrease cancer immunosurveillance. An immunocompromised person may be particularly vulnerable to opportunistic infections, in addition to normal infections that could affect immunocompetent individuals.

The immune condition may be skin-based, such as a skin malignancy.

The method may be used to “treat” an immune condition, where the composition or agents are administered to a subject who already has the immune condition, in order to cure, ameliorate or reduce the severity of at least one symptom associated with the condition and/or reduce or halt the progression of the condition.

The combined inhibition of signalling via PD-1 and via the p38 MAP kinase signalling pathway enhances the immune function of T cells, in particular highly differentiated memory T cells in the subject, thus enhancing the body's natural defences against the disease.

The method may be used to “prevent” an immune condition, where the composition or agents are administered to a subject predicted to be at risk from contracting or developing the immune condition. The method may be used to reduce the likelihood of contracting or developing the immune condition, or, if the immune condition cannot be prevented entirely, at least reducing the severity and/or duration of the immune condition.

The combined inhibition of signalling via a receptor such as PD-1 and via the p38 MAP kinase signalling pathway enhances the immune function of T cells, in particular highly differentiated memory T cells in the subject, thus preparing the immune system to prevent development of the immune condition.

### Vaccination

There is also provided a method for enhancing the immune response to vaccination in a subject by co-inhibiting signalling via a receptor such as PD-1 and via the p38 MAP kinase signalling pathway.

The method may comprise the step of administering an agent capable of inhibiting signalling via a receptor such as PD-1 and an agent capable of inhibiting the p38 MAP kinase signalling pathway to the subject before, during or after administration of a vaccine to the subject.

The combined inhibition of signalling via a receptor such as PD-1 and via the p38 MAP kinase signalling pathway enhances the immune function of T cells, in particular highly differentiated memory T cells in the subject, thus enhancing the immune response to the vaccine.

### Pharmaceutical Composition

The present invention also provides a pharmaceutical composition comprising an agent capable of inhibiting signalling via PD-1, Tim-3, Lag-3 or CTLA-4 and an agent capable of inhibiting the p38 MAP kinase signalling pathway.

The pharmaceutical composition may also comprise a pharmaceutically acceptable diluent, stabiliser and/or excipient.

The pharmaceutical composition may also comprise a vaccine.

The agent capable of inhibiting signalling via PD-1 may be an anti-PD-L antibody, such as an anti-PD-L1 antibody or an anti-PD-L2 antibody or a combination thereof.

The agent capable of inhibiting the p38 MAP kinase may be a small molecule inhibitor such as BIRB796.

### Kit

The present invention also provides a kit comprising:
(i) an agent capable of inhibiting signalling via PD-1, Tim-3, Lag-3 or CTLA-4; and
(ii) an agent capable of inhibiting the p38 MAP kinase signalling pathway for separate, sequential or simultaneous administration.

The kit may also comprise a vaccine, or another pharmaceutical preparation for treatment/prevention of the immune condition.

The kit may also comprise instructions for use.

The present invention also provides a pharmaceutical composition or a kit of the invention for use in enhancing the immune function of a T cell, such as an EMRA T cell.

The present invention also provides the use of (i) an agent capable of inhibiting signalling via PD-1, Tim-3, Lag-3 or CTLA-4; and (ii) an agent capable of inhibiting the p38 MAP kinase signalling pathway in the manufacture of a medicament for use in the treating and/or preventing an immune condition in a subject.

The present invention also provides a method for treating and/or preventing an immune condition in a subject, which comprises the step of administering a pharmaceutical composition or a kit of the present invention to the subject.

The route of administration will depend on the nature of the immune condition and the type of agents/composition. Potentially suitable administration routes include oral, intravenous, transdermal and topical administration.

The long-term blockade of p38 and/or PD-1 signalling is associated with some risks, so the treatment of the present invention may be short-term, in order to enhance immunity temporarily without causing problems associated with excessive T cell activity.

A short-term treatment may be a single treatment or a course of treatments which span less than 14, 10, 5 or 3 days.

### Subject

The subject may be a human or animal subject. The subject may be a mammalian subject.

The subject may be a male or female subject.

In particular the subject may be a middle-aged or elderly human subject, or at least 50, 60, 70 or 80 years of age.

The subject may have, or have a history of an immune condition, such as shingles, pneumonia, skin cancer or an immunodeficiency.

The subject may have a decreased ability to mount a secondary immune response to recall antigens, for example in the skin. The subject may have a significant population of highly differentiated T cells, for example, EMRA CD8+ T cells with reduced proliferative potential.

The invention will now be further described by way of Examples, which are meant to serve to assist one of ordinary skill in the art in carrying out the invention and are not intended in any way to limit the scope of the invention.

### EXAMPLES

### Example 1

### CD45RA⁺CD27⁻ CD8⁺ T Cells Exhibit Characteristics of Senescent T Cells

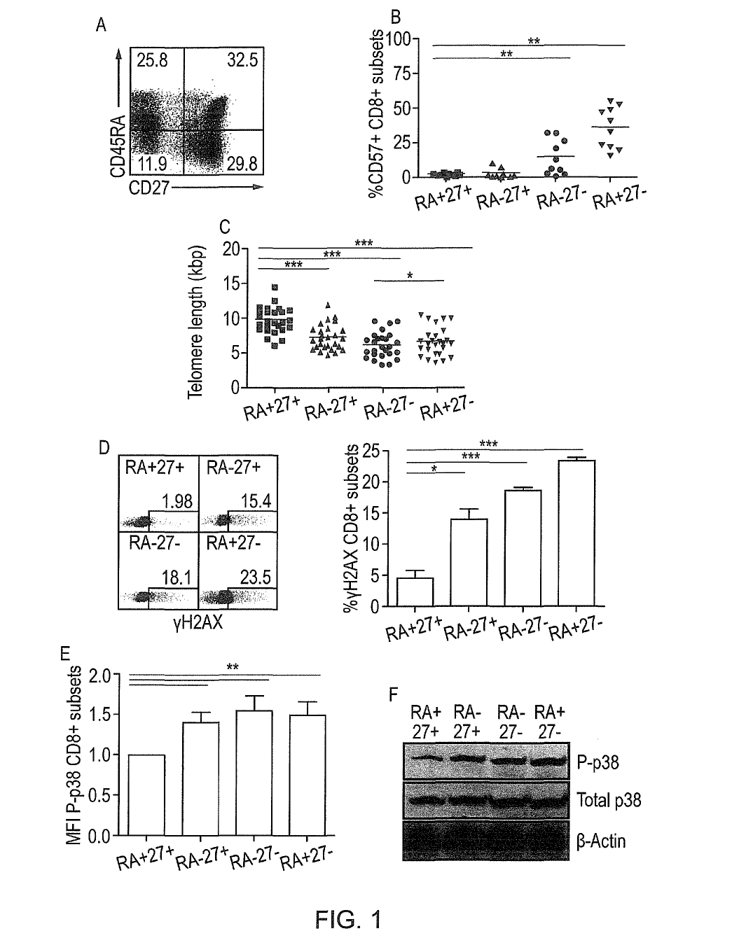

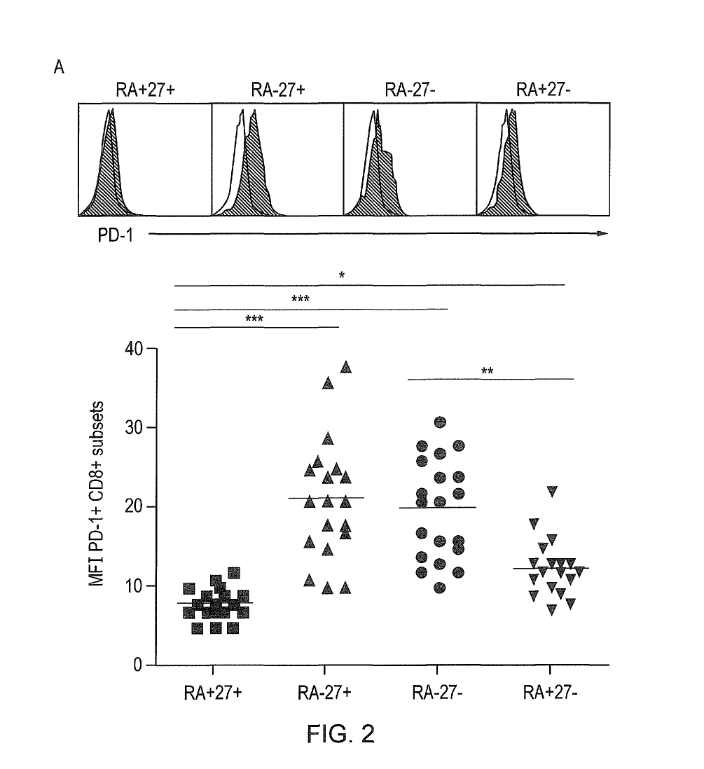

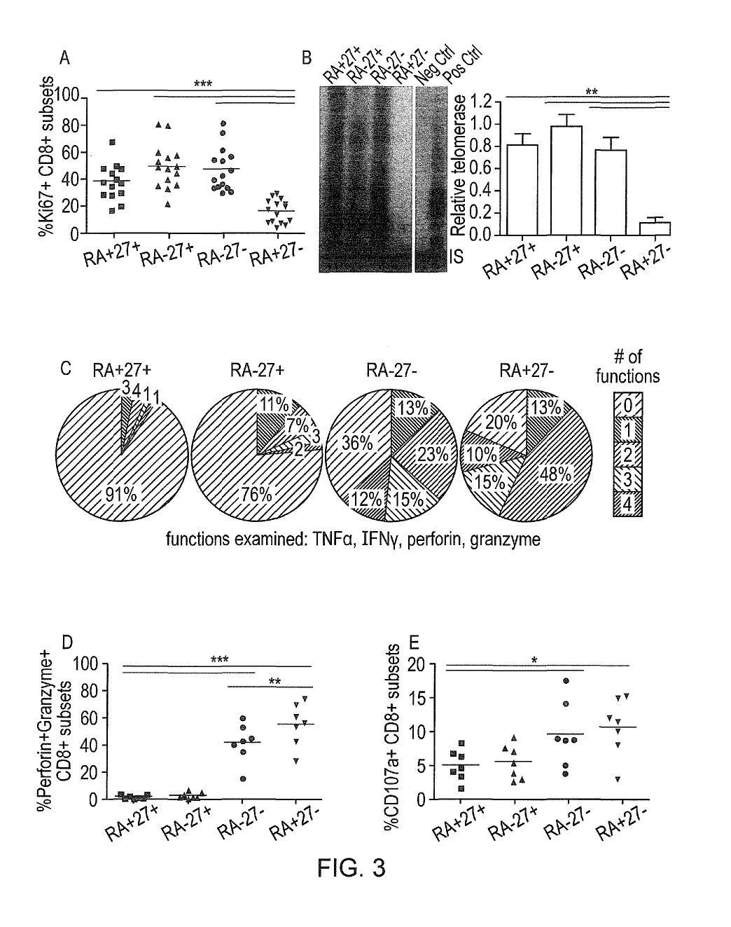

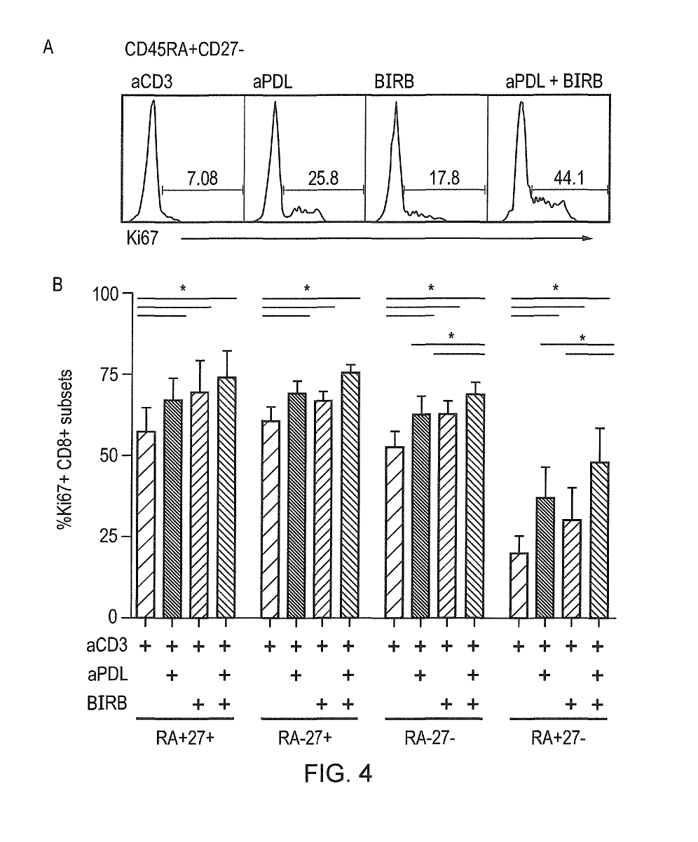

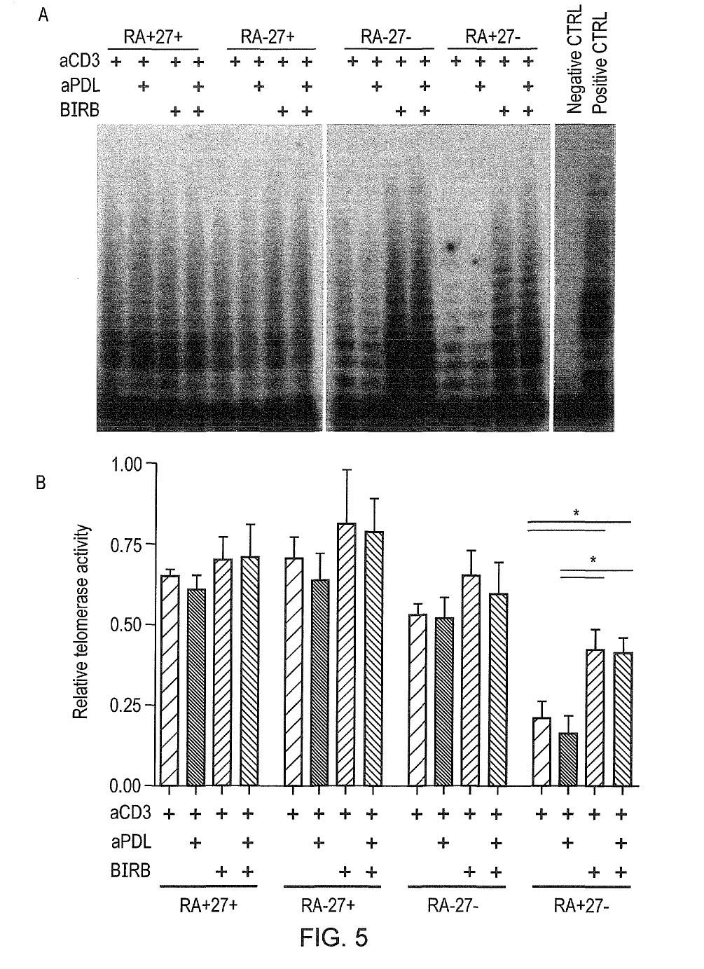

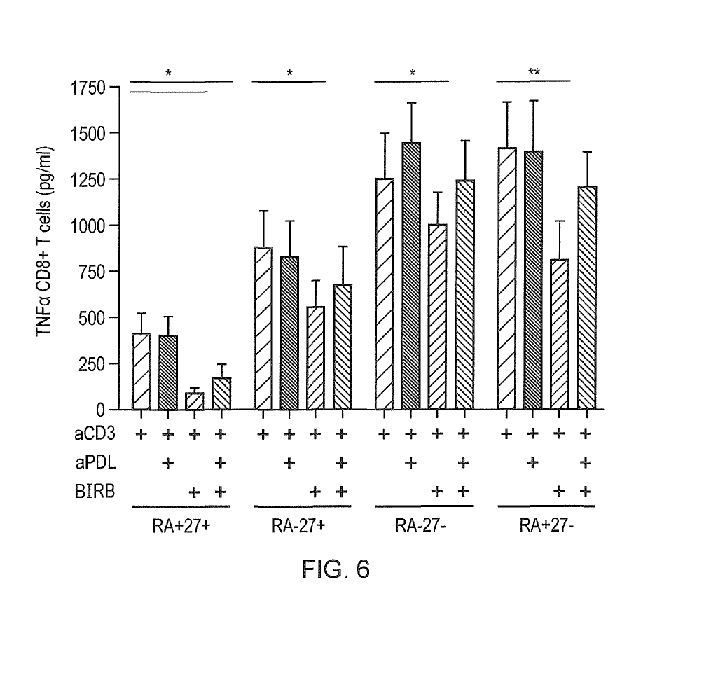

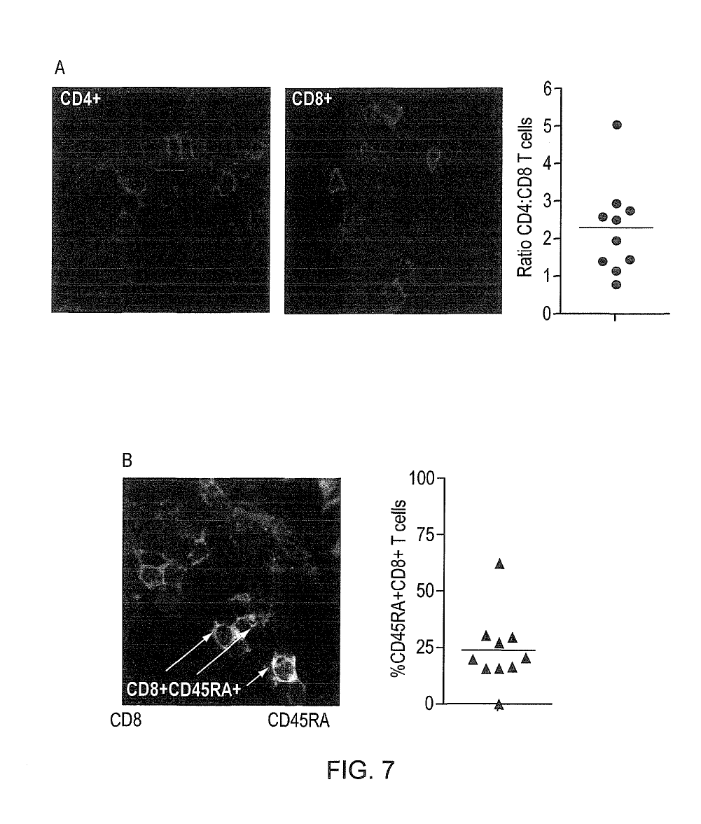

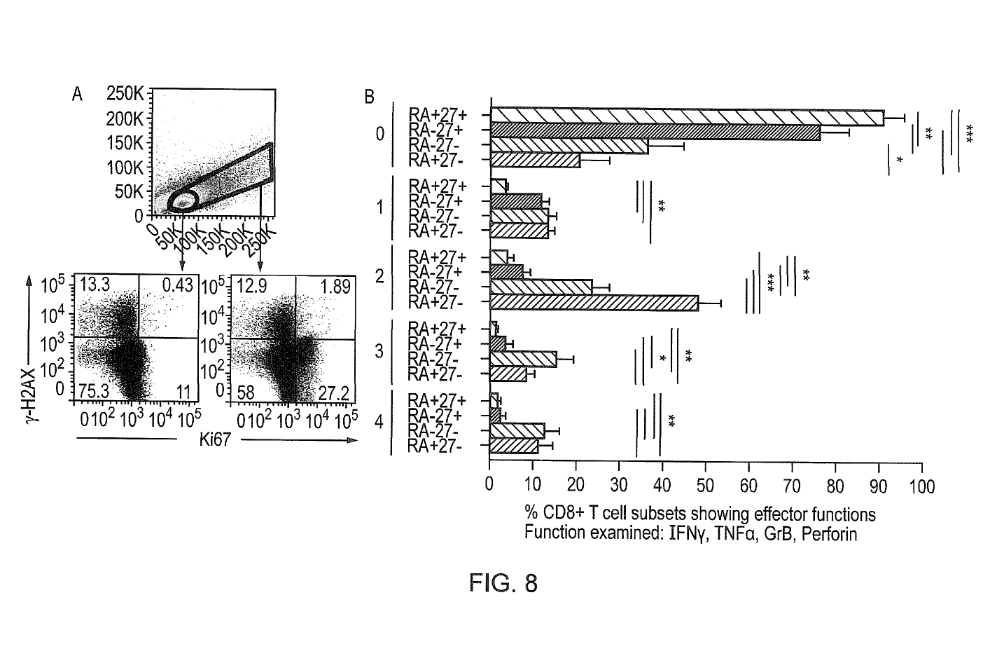

Human CD8⁺ T cells can be subdivided into 4 populations on the basis of their relative surface expression of CD45RA and CD27 molecules (FIG. 1A). Four subsets can be defined, namely naïve (N: CD45RA⁺CD27⁺); central memory (CM: CD45RA⁻CD27⁺); effector memory (EM: CD45RA⁻CD27⁻); and effector memory T cells that re-express CD45RA (EMRA: CD45RA⁺CD27⁻). These subsets are analogous to those identified in other reports where surface CCR7 together with CD45RA expression were used to distinguish between T cells at different stages of differentiation. The present inventors found that CD45RA⁺CD27⁻ CD8⁺ T cells express significantly greater levels of surface CD57, that defines highly differentiated and/or senescent T cells compared to the other subsets (FIG. 1B; P<0.001). In addition it was found that the EM and EMRA populations had significantly shorter telomeres than the N and CM subsets indicating that the former had experienced more cycles of proliferation than the latter (FIG. 1C). However the EMRA subset had significantly longer telomeres than the EM subset, confirming previous reports in CD4⁺ T cells defined using the same markers (Di Mitri et al., 2011, as above). The shortening of telomeres triggers a DNA damage response that can be quantified by antibody staining for phosphorylatedH2AX□H2AX), a member of the histone H2A family that is phosphorylated in response to double-strand breaks. It was found that after activation with anti-CD3 antibody and IL-2, there was a progressive increase of □H2AX staining with the naïve cells expressing lowest and the EMRA population expressing the highest levels of this molecule (FIG. 1D). This increased γH2AX expression was due to DNA damage and not DNA replication as proliferating (Ki67⁺) CD8⁺ T cells did not express this molecule (FIG. 8A). Increased expression of p38 MAPK is also an indicator of senescence. A progressive increase of phosphorylated p38 MAPK was found following stimulation as determined by flow cytometry, with naïve T expressing the lowest and EMRA T cells expressing significantly higher levels of this molecule relative to the naïve cells following stimulation (FIG. 1E; p<0.005). Furthermore, this elevated expression of phosphorylated p38 MAPK was also observed directly ex vivo by Western blot (FIG. 1F). Collectively these results indicate that in terms of phenotypic characteristics, both EM and EMRA CD8⁺ T cells have characteristics of highly differentiated cells that are close to an end-stage or senescence.

### Example 2

### Expression of PD-1 During CD8⁺ T Cell Differentiation

PD-1 is the most investigated inhibitory receptor that is expressed by exhausted CD8⁺ T cells. However, little is known about how the expression of this molecule changes during human T cell differentiation. When the expression of PD-1 on CD45RA/CD27 defined CD8⁺ T cell subsets was examined, the highest level of expression was found to be on the CM (CD45RA⁻CD27⁺) and EM (CD45RA⁻CD27⁻) subsets (FIG. 2). The EMRA population expressed significantly higher levels of this molecule than naïve CD8⁺ T cells but lower levels of this molecule the CM and EM cells (FIG. 2). These results suggest that, based on PD-1 expression, the EMRA subset is unlikely to be an exhausted population.

### Example 3

### Differentiation-Related Functional Changes in Human CD8⁺ T Cells

The proliferative activity of isolated CD8⁺ T cells was investigated at different stages of differentiation. The EMRA population T cells showed significantly less proliferative activity after activation, as defined by Ki67 expression, compared to the other subsets (FIG. 3A; P<0.0001). Highly differentiated CD8⁺ T cells were found to have low telomerase activity and low telomerase activity was found to be confined to the EMRA and not the EM subset (FIG. 3B).

The effector capability of CD8⁺ T cells was investigated at different stages of differentiation using multi-parameter flow cytometry to analyse the expression of TNFα, IFNγ, perforin and granzyme B after anti-CD3 stimulation (FIG. 3C). It was found that, despite the decreased proliferative function and low telomerase activity, the highly differentiated CD45RA⁺CD27⁻ T cells were more multifunctional than the other subsets and contained significantly more cells that expressed 2 functions compared to naïve, CM and EM cells (FIG. 3C and FIG. 8B; P<0.0005). Furthermore, the EMRA population expressed the highest proportion of perforin and granzyme double positive cells compared to all the other subsets (FIG. 3D). In addition, CD107a, a marker of CD8⁺ T cell cytotoxic degranulation following stimulation, was also highly expressed on CD45RA⁺CD27⁻ T cells when compared to the less differentiated CD45RA⁺CD27⁺ and CD45RA⁻CD27⁺ T cell subsets (FIG. 3E). These data suggest that CD45RA⁺CD27⁻ T cells are potent effector cells despite their low proliferative potential.

### Example 4

### Signalling Through Both PD-1 and p38 Pathways Contribute to Reduced Proliferation the of CD45RA⁺CD27⁻ (EMRA) T Cells

It was investigated whether either PD-1 or p38 signalling pathways contributed to the decreased proliferative activity of EMRA CD8⁺ T cells. The four CD45RA/CD27 defined T cell subsets were isolated and then stimulated with anti-CD3 antibody and irradiated autologous APCs in the presence of either 10 μg/ml PDL1/2 blocking antibodies, 500 nM BIRB796 or both inhibitors together. Proliferative activity was determined by staining with Ki67 antibody. Representative histograms for the EMRA subset with and without the inhibitors is shown in FIG. 4A, while pooled results from all four subsets from 4 separate donors is shown in FIG. 4B. It was found that inhibiting both PD-1 and p38 signalling separately increased proliferative activity in CD8⁺ T cells at all four stages of differentiation. Furthermore, there was an additive enhancement when both signalling pathways were inhibited together (FIGS. 4A, 4B). The most striking observation, however, was in the EMRA subset where the proliferative defect could be substantially reversed when both inhibitors were used together, suggesting that the defective proliferative activity of EMRA T cells is actively maintained by senescence as well as exhaustion-related signalling pathways.

### Example 5

### Signalling Through p38 but not PD-1 Pathways Regulates Reduced Telomerase Activity of CD45RA⁺CD27⁻ (EMRA) T Cells

It was next investigated whether p38 or PD-1 signalling were involved in the decreased telomerase activity in EMRA CD8⁺ T cells. Previous data indicated that p38 was involved in the down regulation of telomerase activity in CD4⁺ EMRA T cells (Di Mitri et al., 2011 as above). Once again, CD8⁺ T cell subsets were stimulated with anti-CD3 and irradiated autologous APCs in the presence of both inhibitors separately or together as in FIG. 4. It was found that incubation with BIRB caused a significant increase in telomerase activity in the CD45RA⁺CD27⁻ population (p<0.05) either when added alone or with anti PDL-1/2 (FIG. 5A, representative experiment; FIG. 5B pooled results from 4 different donors). The blockade of PD-1 signalling had no effect on telomerase activity in any of the subsets. In addition, although there was a trend towards increased telomerase activity in N, CM and EM CD8⁺ T cells when p38 signalling was blocked, the results were not significant. These results that suggest that the decreased telomerase activity in EMRA CD8⁺ T cells these cells is maintained actively by p38 signalling. Although these cells express significantly higher levels of PD-1 than the naive population that is sufficient to regulate proliferative activity (FIG. 4), PD-1 blockade either alone or in combination with p38 has no role in regulating telomerase activity in these cells.

### Example 6

### Signalling Through p38 Inhibits TNF-α Secretion that is Reversed by Inhibiting PD-1 Signalling in CD8⁺ T Cell Subsets

p38 signalling plays an essential role in the production of TNF-α. In this example it was found that EM and EMRA CD8⁺ T cells secreted the highest levels of this cytokine after activation (FIG. 6). However, the inhibition of p38 signalling significantly inhibited the secretion of this cytokine in all four subsets. It was found that the inhibition of PD-1 signalling did not increase TNF-α secretion in any of the subsets which was expected as none of these cells were not exhausted per se. However, the blockade of PD-1 signalling prevented the significant downregulation of cytokine secretion in all four subsets that resulted from blocking p38. Collectively these results suggest that the simultaneous blockade of p38 and PD-1 signalling pathways together is better than blocking either pathway alone to enhance the functional activity of EMRA CD8⁺ T cells. This is because blocking of both pathways together enhances proliferative activity and continuous replicative capacity EMRA CD8⁺ T cells through telomerase induction while allowing them to retain their capacity for pro-inflammatory cytokine secretion.

### Example 7

### CD45RA⁺CD27⁺ CD8⁺ T Cells Account for a Large Proportion of Resident Skin T Cells

The results of the previous Examples have been on EMRA CD8⁺ T cells that were isolated from peripheral blood. Their phenotypic characteristics of an end stage cells suggests that they may be en route to be cleared in the liver or spleen. Alternatively, their highly potent effector function suggests that these cells may be part of a first line of defence against antigenic re-challenge. To clarify this further, it was investigated whether these cells are found at a site of frequent immune stimulation such as the skin. T cells in the skin act as a first line of defence against foreign antigens and infective agents. The present inventors investigated whether EMRA CD8⁺ T cells could contribute to this cutaneous immune barrier. Immunohistological analyses of 5 mm punch skin biopsies taken from sun protected forearms of healthy adult volunteers were performed. Immunofluorescence staining of tissue sections showed a higher proportion of CD4⁺ than CD8⁺ T cells in normal skin (FIG. 7A). Furthermore, 25% of the CD8⁺ T cells express CD45RA (FIG. 7B). Previous studies have estimated that there are 2×10¹⁰ T cells in the entire skin surface, nearly twice the number of T cells in circulation. Extrapolating from the relative proportions of CD4 and CD8 cells in skin that were observed (FIG. 7C; ˜2.3:1 ratio of CD4 to CD8 T cells) it is estimated that there are 5×10⁹ re CD8⁺ T cells in the skin. Since CD45RA⁺CD8⁺ T cells comprise 25% of the total T cell population we estimate that there are 1.25×10⁹ of these cells in the skin. These CD45RA⁺CD8⁺ T cells are not recently activated cells, or populations that are in the process of undergoing apoptosis as we found no evidence of either proliferation (Ki67) or cell death (Annexin V) staining in skin T cells (data not shown). These results support the prediction that EMRA CD8⁺ T cells may have a role at sites of intense immune activation or surveillance.

### SUMMARY

The CD8⁺ EMRA T cell population is a potent effector subset that has its replicative potential and survival capacity rigidly controlled by non-overlapping senescence- and exhaustion-related signalling pathways. These cells are predominantly found at peripheral tissue sites such as the skin where there is frequent exposure to foreign antigens and may function as a sentinel population of effector cells. The simultaneous targeting of both the p38 (senescence) and PD-1 (exhaustion) pathways enhances their proliferative responses whilst allowing the retention of effector capacity. p38 inhibitors can be used for immune enhancement, providing that PD-1 signalling is inhibited at the same time.

The greatest functional effect of PD-1 and p38 inhibition was observed in the CD8⁺ T cell EMRA subset, where p38 is maximally expressed but PD-1 is not.

The augmented levels of DNA damage observed in EMRAs may be a consequence of their highly impaired telomerase expression since telomerase can protect cells against oxidative stress and is a critical regulator of the DDR. p38MAPK is activated in response to DNA damage and indeed, its expression on different CD8⁺ T cell subsets correlates with the phosphorylation of □H2AX, a component of DNA damage foci. Although p38 signalling has a central role in inducing senescence that results in apoptosis of the cells, it is also involved in pro-inflammatory cytokine production by CD4⁺ and CD8⁺ T cells including IFN-γ and TNF-α production. This suggests that p38 signaling may be a key component that regulates the development of typical effector cell characteristics such as susceptibility to death but potent functional capacity.

### Materials and Methods

Blood Sample Collection and Isolation Heparinised peripheral blood samples were taken from healthy volunteers (Age range: 20-41, median 30 years). All samples were obtained in accordance with the ethical committee of Royal Free and University College Medical School. Peripheral blood mononuclear cells (PBMC) were isolated using Ficollhypaque (Amersham Biosciences) and either analysed immediately or cryopreservered.
Flow Cytometric Analysis and Cell Sorting Five colour flow cytometric analysis was performed using the following antibodies: PE-conjugated PD-1 (EH12-2H7), CD8 PerCP (SK1), CD27 FITC (M-T271), CD27 APC-H7 (M-T271), CD45RA PE-Cy7 (L48), CD57 PE (X), IFNγ V450 (B27), granzyme B Alexa Flour 700 (GB11), TNFα PE (MAb11), perforin FITC (δG9), CD107a APC (H4A3) all from BD Biosciences. PBMCs were stimulated with 0.5 μg/ml plate coated anti-CD3 (OKT3) at 37° C. for 48 hrs to enable PD-1 expression. For intranuclear staining the following antibodies were used: Ki67 FITC (B56; BD Biosicences), p38 Alexa Fluor 488 (36/p38; BD Pharmingen) and γH2AX Alexa Fluor 488 (2F3, Biolegend). All samples were run using an LSR II (BD Biosciences) and analysed using FlowJo software (Treestar).

CD8⁺ T cells were purified by negative selection using the VARIOMACSsystem (MiltenyiBiotec) according to the manufacturer's instructions. Negatively selected CD8⁺ T cells were labeled with CD27-PE and CD45RA-APC (BD Biosciences) and sorted using a FACSAria (BD Biosciences). The purity of CD8 T cell subsets was assessed by flow cytometry and was always >98%. Multi-parameter flow cytometry was analysed and presentation of distributions was performed using SPICE version 5.2, downloaded from http://exon.niaid.nih.gov. Comparison of distributions was performed using a Student's t test and a partial permutation test.

### Phospho-Cytometry

The analysis of p38 (pT180/pY182) was performed after a 20 minute stimulation with 50 ng/ml PMA and 500 ng/ml ionomycin. Following surface staining for CD45RA, CD27 and CD8, PBMCs were fixed with warm Cytofix Buffer (BD Biosciences) at 37° C. for 10 minutes. Cells were then permeabilized with ice-cold Perm Buffer III (BD Biosciences) at 4° C. for 30 minutes and incubated with the anti-p38 antibody (pT180/pY182) for 30 minutes at room temperature. For the detection of γH2AX (pSer139), purified subsets were activated with 0.5 μg/ml plate coated anti-CD3 and 5 ng/ml of rhlL-2 for 4 days, following which the above staining method was used.

### Western Blot Analysis

Cell lysates were made from sorted CD45RA/CD27 CD8⁺ T cell subsets by sonication in 50 mMTris-HCl (pH 7.5), 2 mM EGTA, 0.1% Triton X-100 buffer. Lysates from 2×10⁵ cells were fractionated by SDS-polyacrylamide electrophoresis and analysed by immunoblotting with phospho-p38 MAPK (12F8, Cell Signalling) using the ECL Advanced Western Blotting Detection Kit (GE Healthcare), according to the protocol provided by the manufacturer.

### The Effect of Cytokine on PD-1 Expression

PBMC were stimulated in duplicate with 0.5 μg/ml plate coated anti-CD3 for 24 hours in the presence of 10 ng/ml IL-15, IL-7, IL-10, IL-6 and 500 U/ml IF N̅□ (PeproTech EC) prior to flow cytometric analysis of PD-1 expression, as detailed above.

### Proliferation Assays

PBMCs or sorted CD8⁺ T cells were stimulated with 0.5 μg/ml plate coated anti-CD3 (OKT3) for 4 days and proliferation was assessed by staining for the nuclear antigen Ki67 that is expressed by cells at all stages of the cell cycle.

### Assessment of Telomere Length

Telomere length of CD8⁺ T cells was measured using a modified version of the flow-FISH method. In brief, PBMCs were washed in PBS and fixed in BS3 (final concentration 1 mM; Perbio Science) for 30 minutes at 4° C. The reaction was quenched using 1 ml of 50 mMTris (pH 7.2) in PBS and incubated in the dark for 20 minutes at room temperature. After washing in PBS followed by hybridization buffer (70% formamide, 20 mMTris, 150 mMNaCl, and 1% BSA), cells were incubated in 0.75 mg/ml of the nucleic acid telomeric probe (CCCTAA)₃ conjugated to Cy5 (Applied Biosystems). Samples were then heated for 10 minutes at 82° C. and left to hybridize in the dark at room temperature for 1 hour. Samples were washed in post hybridization buffer (70% formamide, 10 mMTris, 150 mMNaCl, 0.1% BSA, and 0.1% Tween 20) followed by PBS and analyzed immediately by flow cytometry. Samples were analyzed with and without probe to control for differences in background fluorescence between samples. To ensure consistency of the results between experiments, cryopreserved PBMC samples with known telomere fluorescence and telomere lengths as determined by Southern Blot analysis were used as standards. Results were obtained as median fluorescence intensity values, which could then be converted to telomere length in kilobases using a standard curve. The standard curve was constructed using samples of varying telomere length analyzed both by flow-FISH and telomeric restriction fragment analysis.

### Measurement of Telomerase Activity

Purified CD8⁺ T cell populations (2×10⁵ cells) were snap-frozen after stimulation for 4 days with 0.5 μg/ml plate coated anti-CD3 (OKT3) and irradiated APCs. Telomerase activity was determined using the telomeric repeat amplification protocol (TRAP; TRAPeze telomerase detection kit; Chemicon) according to the protocol provided by the manufacturer. The absolute numbers of CD8⁺ T cells were enumerated using trypan blue (Sigma). The TRAP assay was performed with samples adjusted to 500 Ki67⁺ T cells per reaction to control for the different extents of proliferation in the different subsets after activation.

### PDL-1/2 Blockade and p38 Inhibitor

Signalling through PD-1 on purified total CD8⁺ and CD27/CD45RA defined CD8⁺ subsets was blocked by adding 10 μg/ml each of anti-PD-L1 (29E.2A3.C6) and anti-PD-L2 (24F.10C12.G12), or 10 μg/ml each of IgG2a (Mg2a-53) or IgG2b (MPC-11) isotype control antibodies (Abcam) at the start of the 4 day stimulation period with anti-CD3 (purified OKT3, 0.5 μg/ml) and irradiated APCs. The p38 inhibitor BIRB796 was added to the 4 day cultures at a final concentration of 500 nM and 0.1% DMSO was used as control.

### Measurement of TNFα Following Inhibition

Different CD45RA/CD27 defined CD8⁺ T cell subsets were sorted and 2×10⁵ cells were cultured with 0.5 μg/ml plate coated anti-CD3 (OKT3) and 5 ng/ml rhlL-2, with or without anti-PDL1/2 and BIRB796. Culture supernatants were collected at 48 hours for the measurement of TNF-a using the Quantikine human TNF□ immunoassay (R&D Systems) according to the protocol provided by the manufacturer.

### Histological Analysis of Skin Biopsies

5 mm skin punch biopsies were collected from the volar aspect of the forearm from healthy volunteers. Frozen 5 μm sections were stained using double indirect immunofluorescence. In brief, frozen sections were allowed to come to room temperature, and blocked using a protein block from DakoCytomation. Primary Abs (anti-CD4, anti-CD8 and anti-CD45RA) were incubated overnight at 4° C. in the dark. Secondary fluorochrome-conjugated Abs (anti-mouse Alexa488 and strep-Cy3) were added and incubated for 45 min at room temperature in the dark. Slides were washed and mounted with Vectashield (Vector Laboratories). Images were acquired using the appropriate filters on a Leica DMLB microscope with a ×40 objective, in conjunction with a Cool SNAP-Pro cf Monochrome Media Cybernetics camera and ImagePro PLUS 6.2 software. When counting the numbers of cells in perivascular infiltrates, the 5 largest perivascular infiltrates present in the upper and middle dermis were selected for analysis. Cell numbers were expressed as the mean absolute cell number per donor counted within the frame.

### Statistical Analysis

Statistical significance was evaluated using the Student's t test. Differences were considered significant when p was <0.05.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in cell and molecular biology, immunology or related fields are intended to be within the scope of the following claims.

1. A method for enhancing the immune function of a T cell which comprises the step of inhibiting both:
(i) signalling via PD-1, Tim-3, Lag-3 or CTLA-4; and
(ii) the p38 MAP kinase signalling pathway in the T cell.

2. A method according to claim 1, which comprises the step of inhibiting both:
(i) signalling via PD-1; and
(ii) the p38 MAP kinase signalling pathway in the T cell.

3. A method according to claim 1 or 2, wherein the proliferative potential of the T cell is enhanced without significant loss of T cell function.

4. A method according to any preceding claim, wherein the telomerase activity of the T cell is enhanced.

5. A method according to claim 2, wherein PD-1 signalling is inhibited by blocking the PD-1 ligand (PD-L).

6. A method according to claim 5, wherein PD-L is blocked using an anti-PD-L antibody.

7. A method according to any preceding claim, wherein the T cell is a memory T cell.

8. A method according to claim 7, wherein the memory T cell is an effector memory T cell which expresses CD45RA (EMRA T cell).

9. A method according to claim 8, wherein the memory T cell is a CD8+ EMRA T cell.

10. A method for treating and/or preventing an immune condition in a subject, which comprises the step of enhancing the immune function of a T cell in the subject by a method according to any preceding claim.

11. A method for enhancing the immune response to vaccination in a subject which comprises the step of enhancing the immune function of a T cell in the subject by a method according to any of claims 1 to 9.

12. A pharmaceutical composition comprising an agent capable of inhibiting signalling via PD-1, Tim-3, Lag-3 or CTLA-4, and an agent capable of inhibiting the p38 MAP kinase signalling pathway.

13. A kit comprising:
(i) an agent capable of inhibiting signalling via PD-1, Tim-3, Lag-3 or CTLA-4; and
(ii) an agent capable of inhibiting the p38 MAP kinase signalling pathway for separate, sequential or simultaneous administration.

14. A pharmaceutical composition according to claim 12 or a kit according to claim 13 for use in enhancing the immune function of a T cell.

15. A method for treating and/or preventing an immune condition in a subject, which comprises the step of administering a pharmaceutical composition according to claim 12 or a kit according to claim 13 to the subject.

16. A method according to claim 10 or 15, wherein the immune condition is associated with aging.

17. A method according to claim 10, 15 or 16, wherein the immune condition is selected from shingles, pneumonia, skin cancer and an immunodeficiency.
